(19) United States
(12) Patent Application Publication
(10) Pub. No.: US 2026/0118211 A1
(43) Pub. Date: Apr. 30, 2026

(54) METHOD FOR LEAK TESTING OF A HEAT EXCHANGER IN A BLOOD TREATMENT DEVICE

(71) Applicant: B. Braun Avitum AG, Melsungen (DE)

(72) Inventors: Waldemar Janik, Melsungen (DE); Silvie Krause, Melsungen (DE)

(21) Appl. No.: 19/371,209

(22) Filed: Oct. 28, 2025

(30) Foreign Application Priority Data
Oct. 29, 2024 (DE) 10 2024 131 576.8

(51) Int. Cl. G01M 3/32; A61M 1/36

(52) U.S. Cl. CPC G01M 3/3236; A61M 1/3623; A61M 1/367; A61M 2205/15; A61M 2205/3331; A61M 2205/366; A61M 2205/50

(57) ABSTRACT

A method for leak testing of a heat exchanger of a blood treatment device includes monitoring the time curve of a pressure recorded by a pressure sensor in a first section of a fluid circuit of the blood treatment device in an evaluation period, using a computing system to check whether the time curve of the pressure in the evaluation period fulfills one or more predetermined criteria, and determining a leakage of the heat exchanger when the time curve fulfills the one or more predetermined criteria, with the time curve of the pressure in the evaluation period fulfilling the one or more predetermined criteria. Monitoring the time curve in the evaluation period includes monitoring the time curve during a conveyor operation for pressure build-up/negative pressure build-up and/or after a pressure build-up/negative pressure build-up.

### CROSS-REFERENCE TO RELATED APPLICATION

This application claims priority under 35 U.S.C. § 119 to German Application No. 102024131 576.8, filed on October 29, 2024, the content of which is incorporated by reference herein in its entirety.

### FIELD

The present disclosure relates to a method for leakage testing a heat exchanger of a blood treatment device and to a blood treatment device and system comprising the same.

### BACKGROUND

Blood treatment devices, for example dialysis systems, generally use a heat exchanger which uses the heat of the outflowing liquid, for example the dialysate, to preheat the inflowing permeate, which is colder than the outflowing liquid. Leaks in the heat exchanger are not detected in current blood treatment devices. Instead, it is assumed that no leaks go unnoticed.

There is therefore a risk that contamination, for example due to the transfer of outflowing fluid, such as used dialysate, to inflowing fluid, such as fresh permeate, or to the dialysis fluid side, will not be detected.

The present disclosure is based on the task of providing a method that addresses at least some of the problems described above, in particular enabling reliable detection of leaks in the heat exchanger.

### SUMMARY

The present disclosure provides a method, in particular a computer-implemented method, for leakage testing a heat exchanger of a blood treatment device, a blood treatment device and a system.

The present disclosure relates to a, in particular computer-implemented, method for leakage testing a heat exchanger of a blood treatment device, comprising: monitoring the time curve of a pressure recorded by means of a pressure sensor in a first section of a fluid circuit of the blood treatment device in an evaluation period; determining by means of a computing system whether the time curve of the pressure in the evaluation period fulfills one or more predetermined criteria; and determining a leakage of the heat exchanger in the event that the time curve fulfills the one or more predetermined criteria.

The heat exchanger has two flow paths comprising a first flow path fluidically connected to the first section of the fluid circuit and a second flow path fluidically connected to a second section of the fluid circuit. The fluid circuit is connected to an inlet and an outlet via the flow paths.

The first section of the fluid circuit is fluidically separated from the second section of the fluid circuit, the inlet and/or the outlet during the evaluation period using one or more switching elements.

The section of the fluid circuit that is connected to the inlet is referred to as the inlet-side section of the fluid circuit. The area of the fluid circuit that is connected to the outlet is referred to as the outlet-side area of the fluid circuit. The first section, i.e. the section in which the pressure is monitored, may be in the inlet-side or in the outlet-side region.

In the present disclosure, it is often mentioned that the first section or the second section is/are fluidically separated from the respective other section, the inlet and/or the outlet. Unless otherwise specified herein, the first section and the second section are also then respectively fluidically connected to the first flow path and the second flow path of the heat exchanger.

In the present disclosure, it is assumed that the fluidic connection is open or not disconnected for fluidically interconnected areas/sections, unless explicitly stated otherwise.

Monitoring the time curve in the evaluation period comprises monitoring the time curve in a first evaluation period during a conveyor operation for pressure build-up/negative pressure build-up in the first section and/or the second section of the fluid circuit and/or monitoring the time curve in a second evaluation period after a pressure build-up/negative pressure build-up in the first section of the fluid circuit.

In other words, a method can be provided in which the leak tightness of a heat exchanger is systematically checked, in particular whether there is a leak in which outflowing liquid overflows into the inflowing liquid. For this purpose, a pressure sensor is used which monitors the pressure in a specific section.

If a pressure or negative pressure is built up at certain points in the fluid circuit due to conveyor operation, this will not behave as expected if there is no complete fluidic separation between the first and second sections in the area of the heat exchanger due to a leak in the heat exchanger. The same applies after a pressure or negative pressure has built up, for example if the pressure value drops or rises unusually sharply and/or quickly.

The leak can be detected at various sections in the fluid circuit, which are specified in more detail, in particular by way of example, in the preferred embodiments.

For example, fluid may be conveyed from the inlet through the heat exchanger and one or both sections, but in any case into the first section. For this purpose, if the first section is on the inlet side, the first section can be separated from the downstream second section. If the first section is on the outlet side, the first section can be separated from the outlet (after the heat exchanger). This builds up pressure (at least) in the first section. However, if part of the supplied fluid returns to the fluid circuit on the inlet side due to a leaking heat exchanger, the pressure will certainly build up more slowly.

In another example, after pressure has built up in the first section, the first section can be separated from the second section, for example by means of barrier elements, and can be or remain separated from the outlet or inlet. In this way, the first section (together with the first flow path of the heat exchanger) is essentially fluidically isolated when the heat exchanger is sealed. The pressure in the first section will drop faster and/or more in the case of a leaky heat exchanger than in the case of a tight heat exchanger, because fluid will leak from the first section into the second section.

In another example, the first section and the second section may be separated from each other and pressure may be built up in the second section. This should have essentially no effect on the first section that is separated from it. However, if the heat exchanger is leaking, the pressure there will still change.

The fluid circuit can be divided into at least two sections in such a way that the sections can only be completely separated from each other fluidically if the heat exchanger is tight.

As regards terminology, it should be noted here that in the present disclosure, the section in which the course of the recorded pressure is monitored is always referred to as the "first section". This means that the first section depends on where in the fluid circuit the monitoring takes place. Thus, there can also be several first sections if monitoring takes place in several sections. For example, several criteria can also be used, which is described in detail below.

The first section can therefore be the section that is completely or partially fluidically separated and in which the pressure is measured. Depending on the evaluation period and embodiment, this can be different areas of the fluid circuit, in particular an inlet-side or an outlet-side area.

In particular, the tightness can be the tightness between a first flow path in the heat exchanger, for example a flow path of the inflowing liquid, and a second flow path in the heat exchanger, for example a flow path of the outflowing liquid. If the seal is not tight, i.e. there is a leak, liquid can be exchanged between the first and second flow paths. In particular, outflowing liquid can overflow into the flow path of the inflowing liquid. This scenario is extremely critical for blood treatment devices, as harmful substances can reach a patient. In dialysis, for example, efficiency is also reduced.

The leakage test enables reliable detection of leaks, in particular the leak just described.

In normal operation, the fluid is transported in such a way that it flows into a fluid circuit of the blood treatment device through the heat exchanger and flows out of the fluid circuit through the heat exchanger.

As described above, the heat exchanger has two flow paths comprising a first flow path and a second flow path. These are fluidically separated from each other when the heat exchanger is sealed. Heat exchanging can take place between liquid in the first flow path and liquid in the second flow path. If the heat exchanger is tight, this takes place without the liquids in the two flow paths coming into contact with each other. In the event of a leak, liquid is exchanged between the two flow paths.

The flow paths in the heat exchanger can be designed in such a way that heat can be exchanged between the inflowing and outflowing liquid. In normal operation, the inflowing liquid in particular can be colder than the outflowing liquid, so that heat is transferred from the outflowing liquid to the inflowing liquid in the heat exchanger. However, a temperature difference or heat exchanger does not necessarily have to be present during the leakage test.

For example, the heat exchanger may be a tubular heat exchanger, a plate heat exchanger, a spiral heat exchanger, a shell and tube heat exchanger, a shell and tube heat exchanger, or hybrids thereof, although the disclosure is not limited to these examples.

The first flow path is fluidically connected to the first section of the fluid circuit. The second flow path is fluidically connected to a second section of the fluid circuit. The fluid circuit is connected to an inlet and an outlet via the flow paths.

This means that if the first and second sections of the fluid circuit are fluidically separated, for example by a barrier element such as a valve, fluid exchange between the first and second sections can only occur in the event of a leak in the heat exchanger.

It should be noted here that "fluidically connected" in the present disclosure is also intended to include that there is basically a connection that can be shut off, for example by a barrier element, such as a valve, that can be shut off/separated. If such a connection is shut off, for example by a barrier element, then the present disclosure refers to "fluidically separated".

The liquid can be transported, for example, by means of one or more pumps, although other transport methods are also conceivable.

Monitoring the time curve of the pressure recorded by means of the pressure sensor may comprise recording and optionally processing measured values at several time points by means of the pressure sensor. The processing may include, for example, deriving the property from the measured values, pre-processing raw measured values, and/or determining a function representing the time course of the pressure.

According to the present disclosure, the monitoring of the time curve may be performed in a first evaluation period during a conveyor operation for pressure build-up/negative pressure build-up in the first section and/or second section. Alternatively or additionally, the monitoring of the time curve in a second evaluation period may comprise after a pressure build-up/negative pressure build-up in the first section of the fluid circuit. The evaluation period may comprise the first and/or the second evaluation period.

In principle, the evaluation period can be selected according to the circumstances. For example, the evaluation period, in particular the first evaluation period, may comprise only part of a pressure build-up phase or vacuum build-up phase or the entire pressure build-up phase or vacuum build-up phase. Alternatively or additionally, the evaluation period, in particular the second evaluation period, may begin immediately after the pressure build-up/negative pressure build-up or with a certain delay. The evaluation period may also comprise an end of a pressure build-up phase or vacuum build-up phase and the subsequent period.

As mentioned above, the first section of the fluid circuit is fluidically separated from the second section of the fluid circuit, the inlet and/or the outlet during the evaluation period using one or more switching elements.

In addition to switching elements, the fluid system itself can also contribute to the fluidic separation. In the case of a sealed heat exchanger, the fluidic separation between the two flow paths in particular also contributes to the fluidic separation of the first section from the second section of the fluid circuit, the inlet and/or the outlet.

As seen above, during the evaluation period, the first section is at least partially fluidically isolated (from the second section and/or, depending on whether it is inlet or outlet side, from the inlet or outlet). This leads to a very accurate prediction/expectation of how the pressure in the first section should behave during the evaluation period if the heat exchanger is tight. If the heat exchanger leaks, the first section will no longer be separated to the same extent as with a tight heat exchanger. Thus, a leak can be easily and accurately deduced from a deviation from the expectation.

As will be explained in detail below using the examples, this fluidic separation can occur in relation to different areas, for example depending on where and/or when the pressure sensor monitors the pressure.

The pressure sensor may be any suitable known pressure sensor that detects pressure, for example directly or indirectly.

As seen above, the method comprises checking whether the time curve of the pressure in the evaluation period fulfills one or more predetermined criteria. The criteria may include that a rise of pressure and/or loss of pressure is stronger or weaker (e.g. steeper and/or by a larger amount or flatter and/or by a smaller amount) compared to a course without a leak. Detailed examples of the criteria are presented below.

As will also be seen from the description of the examples below, these criteria may indicate a leak, in particular a spill of outflowing liquid into the inflowing liquid.

According to the present disclosure, the method comprises determining a leak of the heat exchanger in the event that the time curve meets the predetermined criteria.

Determining the leak can optionally trigger an output of a warning and/or an automatic initiation of safety measures.

If the leak is determined (only) in the event that several criteria are met, these criteria may relate to the time curve of the pressure in a single first section, for example a time curve during the pressure build-up/negative pressure build-up and a time curve after the pressure build-up/negative pressure build-up or several criteria relating to the curve, for example slope of the pressure change and absolute pressure change. The criteria may alternatively or additionally refer to the pressure in various first sections, which may be monitored by several sensors, for example.

The use of multiple criteria can have various advantages depending on how the criteria are used. For example, it may allow for redundancy, for example in that it is sufficient that only a part, for example only one, of the criteria is met to detect a leak. The robustness of the leakage test can be increased, for example by only detecting a leak if several criteria are met, in particular all criteria. Safety can be increased if a leak is only positively determined if none of the criteria are met. This can be an additional function in addition to determining a leak.

The criteria are predefined and can be derived, for example, from predicted curves and/or empirically determined curves, in particular in comparison to the curves for a tight heat exchanger. Examples can be found below. These may be case-dependent criteria that the person skilled in the art will adapt appropriately in individual cases, especially if quantitative criteria are used that depend on the system design.

The computing system may comprise one or more computing devices, in particular may be a distributed computing system. The computing system may comprise local computers and/or servers and/or mobile user terminals.

In an aspect of the present disclosure, the first section of the fluid circuit may be fluidically separated from the second section of the fluid circuit and the outlet in the evaluation period using one or more switching elements. In particular, the second section may be fluidically connected to the inlet.

In particular, the first section can be arranged between the second section and the outlet and be fluidically separated from both, whereby the first section is still fluidically connected to the first flow path of the heat exchanger, i.e. the shut-off is downstream of the heat exchanger in the direction of flow. In the case of a sealed heat exchanger, the first section would therefore be completely isolated together with the first flow path of the heat exchanger.

In this aspect, monitoring can be performed in the second evaluation period. For example, the monitoring may be performed after the pressure build-up or negative pressure build-up. The predetermined criterion may comprise that a loss of pressure or rise of pressure of the recorded pressure in the second evaluation period deviates from a reference loss of pressure or reference rise of pressure, wherein the loss of pressure or rise of pressure is in particular greater than the reference loss of pressure or reference rise of pressure. In particular, the amount or the slope of the loss of pressure or rise of pressure can deviate from the reference loss of pressure or rise of pressure.

In a tight heat exchanger, the pressure would drop in a certain way after the pressure build-up, for example with a certain gradient and/or by a certain amount. If there is a leak in the heat exchanger, the loss of pressure would be different, in particular it would drop faster or by a greater amount. This also applies analogously to the rise of pressure after a negative pressure has been built up. The pressure or negative pressure is maintained for less time, for example.

In other words, despite insulation, the pressure would change unexpectedly quickly and/or significantly. This is an indication of a leak in the heat exchanger.

Alternatively or additionally, in this aspect, the monitoring in the first evaluation period may be performed during a conveyor operation of a first conveyor arranged in the first section and/or a second conveyor arranged in the second section for pressure build-up/negative pressure build-up in the first section. The predetermined criterion may comprise that a rise of pressure/loss of pressure of the recorded pressure in the first evaluation period deviates from a reference rise of pressure/reference loss of pressure, wherein the rise of pressure/loss of pressure is in particular smaller than the reference rise of pressure/reference loss of pressure.

Similar to what is described for the pressure rise or loss of pressure after the pressure build-up or negative pressure build-up, a certain reference rise of pressure or reference loss of pressure is to be expected for a tight heat exchanger during the pressure build-up or negative pressure build-up. However, in the event of a leak, for example, the pressure or negative pressure can be built up less efficiently, resulting in a deviation, in particular a slower build-up of pressure or negative pressure.

This means that a leak can be detected easily and reliably.

The pressure build-up/negative pressure build-up described above can take place when the first and second sections are fluidically connected.

Alternatively, a pressure/vacuum can also be built up in one of the two sections if the two sections are fluidically separated from each other. The pressure/vacuum in the other section should not change (significantly) when the heat exchanger is sealed. If it does, this is an indication of a leak.

According to the present disclosure, the first section of the fluid circuit may be fluidically separated from the second section of the fluid circuit and the outlet during the evaluation period using one or more switching elements.

Thus, in the case of a sealed heat exchanger, the first section can be fluidically isolated. A pressure build-up or negative pressure build-up in the second section will have little or no effect on the first section in a sealed heat exchanger in the configuration described above.

In this configuration, the monitoring in the first evaluation period may be performed during a conveyor operation of a second conveyor arranged in the second section for pressure build-up/negative pressure build-up in the second section. The predetermined criterion may comprise that a rise of pressure/loss of pressure of the recorded pressure (as described above, it is the pressure recorded in the first section) in the first evaluation period deviates from a reference rise of pressure/reference loss of pressure, wherein the rise of pressure/loss of pressure is in particular greater than the reference rise of pressure/reference loss of pressure. In other words, a pressure change in the second section affects the pressure in the first section differently, in particular more, than expected for a tight heat exchanger.

In the above configurations, the first section may be located downstream with respect to the second section.

The conveyor may be arranged upstream or downstream with respect to the pressure sensor. Alternatively or additionally, the first conveyor may be arranged downstream with respect to the pressure sensor. However, an upstream conveyor is also possible.

In a further configuration, the first section of the fluid circuit may be fluidically separated from the second section of the fluid circuit and the inlet in the evaluation period using one or more switching elements and, in particular, the second section may be fluidically connected to the outlet. In other words, in this case, the first section may be arranged upstream of the second section in the direction of flow, in particular between the inlet and the second section. However, the above considerations, features and advantages for the configuration in which the second section is arranged upstream of the first section in the direction of flow can be transferred analogously. Ultimately, the only thing that is reversed is which of the heat exchanger's flow paths the respective section is connected to.

In this configuration, monitoring can be carried out in the second evaluation period and the predetermined criterion can comprise that a loss of pressure/rise of pressure of the recorded pressure in the second evaluation period deviates from a reference loss of pressure/rise of pressure, the loss of pressure or rise of pressure being in particular greater than the reference loss of pressure. In other words, a leak can be detected if the pressure changes more than would be expected for a tight heat exchanger. Please refer to the explanations above.

Alternatively or additionally, the monitoring may be performed in the first evaluation period during a conveyor operation of a second conveyor arranged in the first section for pressure build-up/negative pressure build-up in the first section. The predetermined criterion may comprise that a rise of pressure/loss of pressure of the recorded pressure in the first evaluation period deviates from a reference rise of pressure/reference loss of pressure, wherein the rise of pressure/loss of pressure is in particular smaller than the reference rise of pressure/reference loss of pressure. In other words, a leak can be detected if a pressure build-up or negative pressure build-up does not occur as expected, in particular if it occurs more slowly. Please also refer to the explanations above.

As briefly mentioned above, in this configuration the first section may be arranged upstream with respect to the second section.

In this configuration, the second conveyor may, for example, be arranged upstream with respect to the pressure sensor.

In the second evaluation period described above, the conveyor operation, in particular of the first conveyor and/or the second conveyor, may be set. This means, for example, that no further pressure build-up or negative pressure build-up can occur in the second evaluation period. This reduces the susceptibility to errors and increases reliability because reference pressure build-up or reference loss of pressure have fewer dependencies, for example do not depend on the conveyors and their characteristic curves.

The second conveyor can comprise one or more concentrate pumps, which are designed to feed concentrate into a fluid in the fluid circuit. This is advantageous because an existing component can be used and additional components do not have to be provided for the leakage test.

According to the present disclosure, the first section or the second section can be fluidically separable from the outlet by means of an external barrier element. The external barrier element may be controllable by means of a control device of the blood treatment device and/or by means of an external control device, wherein the external control device is configured to control the external barrier element and optionally the blood treatment device. Such a control system makes it possible to coordinate the various components in such a way that the results of monitoring the pressure can be reliably associated with the actuation of barrier elements and/or conveyors, in particular with precise timing. As a result, the comparison with reference curves is more reliable and a leakage can be detected more reliably.

According to the present disclosure, a time point for opening and/or closing the external barrier element can be communicated from the blood treatment device to the external barrier element by means of a synchronization device. Thus, the barrier element does not have to be controlled separately and the advantage described above in connection with the control device can still be achieved.

The external control device can be designed as an external synchronization device, which in particular receives data from the blood treatment device and performs sequence control based thereon. The external synchronization device may in particular be designed such that it performs the entire sequence control. In particular, the external synchronization device may be designed to determine when the external valve and/or other valves are to be opened or closed and/or when the evaluation period begins.

Optionally, all steps can be performed automatically.

The method may comprise controlling one or more barrier elements and/or one or more conveyors, in particular automatically, for example by means of the control device, so that the sections are fluidically separated according to the above method and, if necessary, a pressure build-up or vacuum release is performed according to the above method.

The present disclosure also provides a blood treatment device.

The blood treatment device comprises:

a fluid circuit and a heat exchanger, the heat exchanger having two flow paths comprising a first flow path fluidically connected to a first section of the fluid circuit and a second flow path fluidically connected to a second section of the fluid circuit, the fluid circuit being connected to an inlet and an outlet via the flow paths;

a pressure sensor configured to detect a pressure in the first section of the fluid circuit of the blood treatment device;

a computing system adapted for monitoring a time curve of the pressure recorded by the pressure sensor in an evaluation period in which the blood treatment device has a test configuration, for checking whether the time curve of the pressure in the evaluation period satisfies one or more predetermined criteria, and for determining a leak of the heat exchanger in the event that the time curve satisfies the one or more predetermined criteria,

wherein the first section of the fluid circuit in the test configuration is fluidically separated from the second section of the fluid circuit, the inlet and/or the outlet using one or more switching elements, and wherein monitoring the time curve in the evaluation period comprises monitoring the time curve in a first evaluation period during a conveyor operation for pressure build-up/negative pressure build-up in the first section and/or the second section of the fluid circuit and/or monitoring the time curve in a second evaluation period after a pressure build-up/negative pressure build-up in the first section of the fluid circuit.

The term "test configuration" may be considered as the configuration of the blood treatment device, in particular the fluidic connections and/or circuits of the blood treatment device, in a test period, which may in particular include or coincide with the monitoring period. The test configuration of the blood treatment device may include at least the fluid circuit configuration.

The conveyor operation for pressure build-up may comprise, for example, operation of a pump.

According to the present disclosure, in a first test configuration of the blood treatment device, the first section of the fluid circuit may be fluidically separated from the second section of the fluid circuit and the outlet using one or more switching elements and, in particular, the second section may be fluidically connected to the inlet.

The blood treatment device can be designed to perform the monitoring in the second evaluation period, and the predetermined criterion can comprise that a loss of pressure or rise of pressure of the recorded pressure in the second evaluation period deviates from a reference loss of pressure or reference rise of pressure, wherein the loss of pressure/rise of pressure is in particular greater than the reference loss of pressure.

Alternatively or additionally, the blood treatment device may be configured to perform the monitoring in the first evaluation period during a conveyor operation of a first conveyor arranged in the first section and/or a second conveyor arranged in the second section for pressure build-up/negative pressure build-up in the first section, and the predetermined criterion may comprise that a rise of pressure/loss of pressure of the recorded pressure in the first evaluation period deviates from a reference rise of pressure/reference loss of pressure, wherein the rise of pressure/loss of pressure is in particular smaller than the reference rise of pressure/reference loss of pressure.

In the first test configuration, the first section may in particular be arranged downstream with respect to the second section and/or the conveyor may be arranged upstream or downstream with respect to the pressure sensor and/or the first conveyor may be arranged downstream with respect to the pressure sensor.

According to the present disclosure, in a second test configuration of the blood treatment device, the first section of the fluid circuit may be fluidically separated from the second section of the fluid circuit and the outlet using one or more switching elements.

The blood treatment device may be configured to perform the monitoring in the first evaluation period during a conveyor operation of a second conveyor arranged in the second section for pressure build-up/negative pressure build-up in the second section, and the predetermined criterion comprises that a rise of pressure/loss of pressure of the recorded pressure in the first evaluation period deviates from a reference rise of pressure/reference loss of pressure, wherein the rise of pressure/loss of pressure is in particular greater than the reference rise of pressure/reference loss of pressure.

In the second test configuration, the first section may in particular be arranged downstream with respect to the second section.

According to the present disclosure, in a third test configuration of the blood treatment device, the first section of the fluid circuit may be fluidically separated from the second section of the fluid circuit and the inlet using one or more switching elements and, in particular, the second section may be fluidically connected to the outlet.

The blood treatment device may be configured to perform the monitoring in the second evaluation period, and the predetermined criterion comprises that a loss of pressure of the recorded pressure in the second evaluation period deviates from a reference loss of pressure, wherein the loss of pressure is in particular greater than the reference loss of pressure.

Alternatively or additionally, the blood treatment device may be configured to perform the monitoring in the first evaluation period during a conveyor operation of a second conveyor arranged in the first section for pressure build-up/negative pressure build-up in the first section. The predetermined criterion may comprise that a rise of pressure/loss of pressure of the recorded pressure in the first evaluation period deviates from a reference rise of pressure/reference loss of pressure, wherein the rise of pressure/loss of pressure is in particular smaller than the reference rise of pressure/reference loss of pressure.

In the third test configuration, the first section may in particular be arranged upstream with respect to the second section. The second conveyor may alternatively or additionally be arranged upstream with respect to the pressure sensor. The conveyor may alternatively or additionally be arranged upstream or downstream with respect to the pressure sensor. The first conveyor can alternatively or additionally be arranged downstream in relation to the pressure sensor.

The blood treatment device can be designed at least for collecting, degassing and/or heating liquid as part of an extracorporeal blood treatment. The blood treatment device can also be designed for dosing concentrates for the production of dialysis fluid and/or for balancing including ultrafiltration. The blood treatment device may comprise a blood pump, a dialyzer, a pressure sensor and/or a tubing system. The blood treatment device may also comprise one or more actuators and/or one or more (further) sensors for performing a dialysis treatment.

The blood treatment device may be configured to perform the method of the present disclosure, in particular as described above.

In particular, the method of the present disclosure may be carried out using the blood treatment device of the present disclosure, in particular as described above.

The present disclosure also relates to a system comprising the blood treatment device according to the present disclosure.

The system may comprise an external barrier element and a control device of the blood treatment device and/or an external control device. The first section or the second section may be fluidically separable from the outlet by means of the external barrier element, in particular wherein the external barrier element is controllable by means of the control device of the blood treatment device and/or by means of the external control device, wherein the external control device is configured to control the external barrier element and optionally the blood treatment device.

Alternatively or additionally, the system may comprise an/the external barrier element and a synchronization device, wherein a time point for opening and/or closing the external barrier element is communicated from the blood treatment device to the external barrier element by means of the synchronization device.

The features and advantages described in connection with the method also apply accordingly to the blood treatment device and the system.

### BRIEF DESCRIPTION OF THE DRAWINGS

Further examples and embodiments are explained below with reference to the figures.

FIG. 1 shows a schematic representation of a method according to the present disclosure;

FIG. 2 shows a schematic representation of a system according to the present disclosure;

FIG. 3 shows a schematic representation of a system according to the present disclosure;

FIG. 4 shows a schematic representation of a system according to the present disclosure; and

FIG. 5 shows a schematic representation of a signal waveform over time.

### DETAILED DESCRIPTION

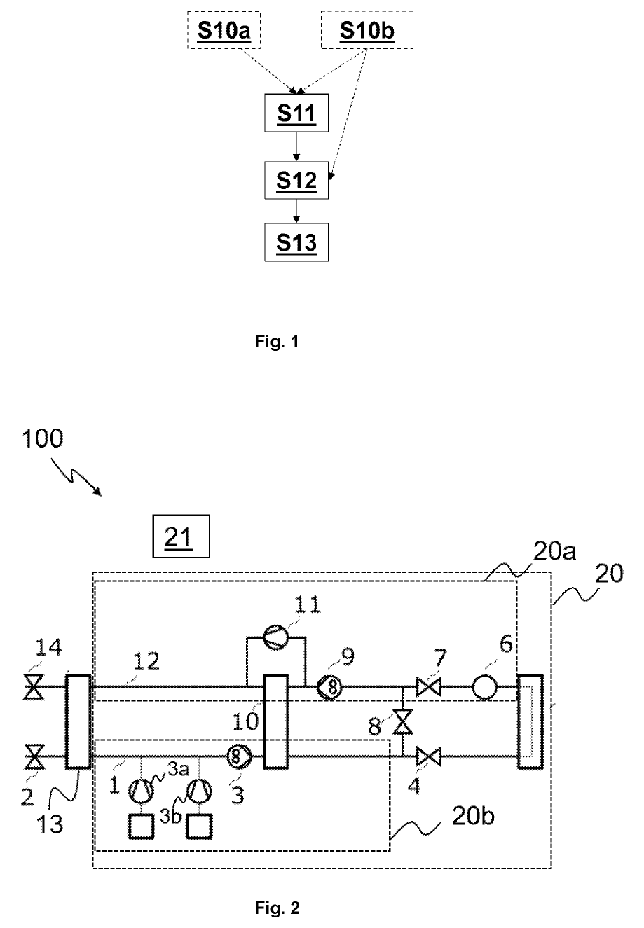

FIG. 1 schematically illustrates a method for leakage testing a heat exchanger of a blood treatment device according to the present disclosure.

The method comprises, in step S11, monitoring the time curve of a pressure recorded by a pressure sensor in a first section of a fluid circuit of the blood treatment device in an evaluation period.

The method comprises, in step S12, checking by means of a computing system whether the time curve of the pressure in the evaluation period fulfills one or more predetermined criteria.

The method comprises, in step S13, determining a leak of the heat exchanger in the event that the time curve fulfills the one or more predetermined criteria.

The heat exchanger thereby has two flow paths comprising a first flow path fluidically connected to the first section of the fluid circuit and a second flow path fluidically connected to a second section of the fluid circuit, and the fluid circuit is connected to an inlet and an outlet via the flow paths.

The first section of the fluid circuit is fluidically separated from the second section of the fluid circuit, the inlet and/or the outlet during the evaluation period using one or more switching elements.

Monitoring the time curve in the evaluation period comprises monitoring the time curve in a first evaluation period during a conveyor operation for pressure build-up/negative pressure build-up in the first section and/or the second section of the fluid circuit and/or monitoring the time curve in a second evaluation period after a pressure build-up/negative pressure build-up in the first section of the fluid circuit.

In optional step S10a, switching elements, for example valves, can be switched to establish the corresponding circuits in the fluid circuit. This can be done automatically. In optional step S10b, delivery elements, for example pumps, can be operated, for example for pressure build-up or negative pressure build-up. This step may, for example, take place before monitoring (for example in the second evaluation period) and/or during monitoring (for example in the first evaluation period).

The method of the present disclosure, in particular described in connection with FIG. 1, can be carried out, for example, with the blood treatment device according to the present disclosure, in particular also as shown in FIG. 2. However, implementation with other blood treatment devices is also conceivable.

FIG. 2 shows an example of a blood treatment device 100 according to the present disclosure.

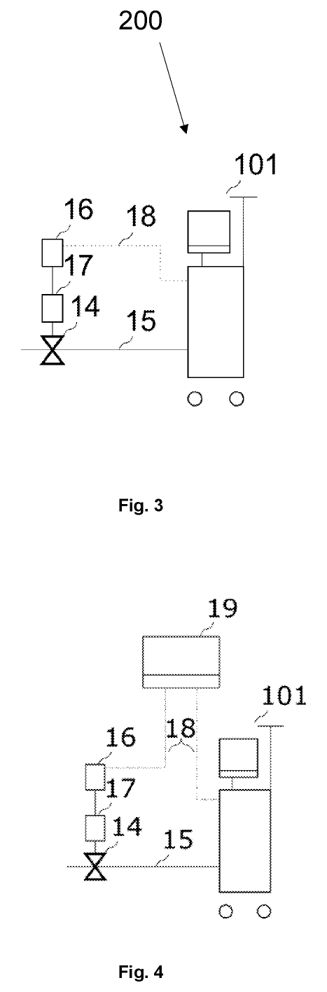

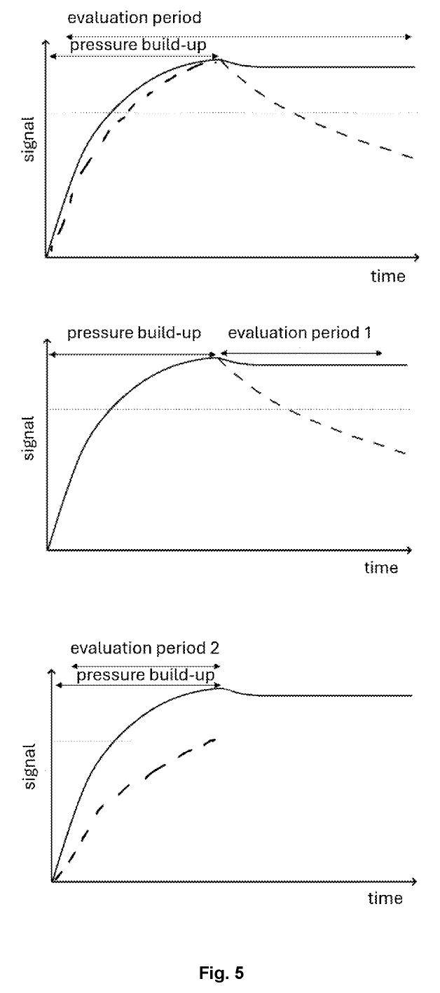

The blood treatment device comprises at least a heat exchanger 13, a fluid circuit 20, a sensor 6 and a computing system 21. The other elements shown in FIG. 2 are optional. The computing system 21 may correspond in whole or in part to the control device 19 in FIGS. 3 or FIG. 4.

The heat exchanger has two flow paths comprising a first flow path fluidically connected to a first section of the fluid circuit, and a second flow path fluidically connected to a second section of the fluid circuit. The fluid circuit is connected to an inlet and an outlet via the flow paths. In the example in FIG. 2, the inlet is still separated from the fluid circuit by a valve 2 (also known as a "water inlet valve" or "inlet valve") and the outlet is separated from the fluid circuit by a valve 14 (also known as a "drain valve" or "outlet valve").

The pressure sensor 6 is configured to detect a pressure in the first section of the fluid circuit 20 of the blood treatment device 100.

The computing system 21 is configured for monitoring a time curve of the pressure recorded by the pressure sensor 6 in an evaluation period in which the blood treatment device has a test configuration, for checking whether the time curve of the pressure in the evaluation period fulfills one or more predetermined criteria, and for determining a leak of the heat exchanger 13 in the event that the time curve fulfills the predetermined criterion or criteria.

In the test configuration, the first section of the fluid circuit is fluidically separated from the second section of the fluid circuit, the inlet and/or the outlet, using one or more switching elements.

Monitoring the time curve in the evaluation period comprises monitoring the time curve in a first evaluation period during a conveyor operation for pressure build-up/negative pressure build-up in the first section and/or the second section of the fluid circuit and/or monitoring the time curve in a second evaluation period after a pressure build-up/negative pressure build-up in the first section of the fluid circuit.

The fluid circuit can, for example, comprise a line 1 on the inlet side to which liquid, for example water, can be supplied via the heat exchanger. A conveying element 3, for example a flow pump, can be provided in the inlet-side area of the fluid circuit for conveying liquid and/or for pressure/negative pressure build-up. Concentrate pumps 3a and 3b can also be provided as an option. These pumps can be used to pump concentrate from corresponding concentrate sources into the fluid circuit, especially in the area of the inlet-side line. These can also be used, if operated appropriately, to pump fluid through the fluid circuit and/or to build up pressure.

The fluid circuit can, for example, comprise a line 12 on the outlet side, from which fluid, for example dialysate, can be discharged from the fluid circuit via the heat exchanger.

The heat exchanger can be designed so that heat is exchanged between the fluid supplied to the fluid circuit through the heat exchanger and the fluid discharged from the fluid circuit through the heat exchanger. If the heat exchanger is sealed, there is no transfer from the outgoing to the incoming liquid or vice versa.

A conveying element 9, for example a flow pump, can be provided in the outlet-side section of the fluid circuit for conveying fluid and/or for pressure/negative pressure build-up.

As shown in FIG. 2, a dialysis bridge/rinsing bridge 5 with an inlet valve 4 (also known as a "dialyzer inlet valve") and an outlet valve 7 (also known as a "dialyzer outlet valve") can be provided between the inlet-side part and the outlet-side part of the fluid circuit. In addition, a valve 8 can be provided which can be used as a bypass, i.e. to divert fluid past the rinsing bridge.

Furthermore, a balancing device 10 and a corresponding conveyor 11 can optionally be provided.

FIG. 2 shows an example of a division into a first section 20a and a second section 20b, whereby in this example the first section is arranged downstream with respect to the second section.

As already explained above, the first and second sections are each dependent on where the pressure is being monitored. Thus, for example, if the pressure sensor 6 were arranged in the area of the line 1 , the first section would be arranged upstream with respect to the second section. In other words, the first section (and correspondingly the second section) may be arranged on the inlet side or the outlet side.

Further advantages and features according to the present disclosure will become apparent from the following description.

The present disclosure describes a method for checking a heat exchanger of a dialysis machine for leaks, wherein a pressure is applied on the dialysis fluid side. Common dialysis machines include a heat exchanger that uses the heat of the outflowing dialysate to preheat the inflowing permeate, which is colder than the dialysate. This heat exchanger is required to be inherently safe. It is therefore assumed that no leaks can occur. In the event of a defect or leak in the heat exchanger, the transfer of used dialysate to the fresh permeate or dialysis fluid side cannot be detected by conventional dialysis machines. This would lead to contamination of the dialysis fluid side, which must be avoided.

Possible methods and devices according to the present disclosure have already been explained above, in particular with reference to FIGS. 1 and 2.

Referring again to FIG. 2, further non-limiting examples are explained in detail below.

FIG. 2 shows a schematic and simplified example of a blood treatment device 100, here using the example of a dialysis system. In the following, an example of the operation of a dialysis system, for example the dialysis system shown in FIG. 2, is briefly described:

High-purity water, so-called permeate, enters the dialysis system through the valve 2, where it passes through the heat exchanger 13. Heat transfer takes place in the heat exchanger 13. The permeate absorbs heat energy from the dialysate, which flows out of the fluid circuit through the line 12 and then through the heat exchanger 13, and is preheated in this way.

The heat exchanger 13 can be located inside or outside a dialysis machine (for example, in relation to a housing).

The permeate continues to flow through line 1.

Optionally, the permeate can flow through a temperature control device (not shown), where the water is heated to the required temperature. The heat exchanger 13 can optionally be designed as an integral part of the temperature control device.

Conveyed by a first conveyor 3, the water or the mixture of water and at least one concentrate flows through the balance device 10 and the dialyzer inlet valve 4 into the rinsing bridge 5. In a dialysis device that is not yet equipped with a dialyzer, this connects the water-side inlet to the outlet instead of a dialyzer. A dialyzer can therefore also be provided instead of the rinsing bridge 5. Alternatively, the water/mixture can be routed through the bypass using a suitable valve position.

The second conveyor 9 conveys the dialysate into the balance device 10, which allows the controlled removal of excess water using the conveyor 11. From there, it passes through the line 12 to the heat exchanger 13 and then into the drain.

The balancing device is optional.

Other common components of blood treatment devices, in particular dialysis systems, such as blood leak detectors, temperature sensors, etc., are not shown in FIG. 2 for the sake of clarity.

In the event of a defective heat exchanger 13, dialysate may flow from the line 12 into the line 1 carrying the permeate or dialysis fluid and contaminate it. Since this would also lead to a reduction in dialysis efficiency if undetected, the present disclosure describes methods for detecting a defect or a leak in the heat exchanger 13.

In a first example, fluid is conveyed into the circuit of the dialysis system 100 using conveyors 3 and 9. The balancing device is in flow mode while conveyor 11 also assumes a defined position. The temperature in the dialysis system 100 is known at all times or is set to a defined value with the aid of the temperature control device. In this embodiment, valve 14 is located inside the dialysis machine and can be closed for the purpose of generating pressure. If fluid continues to be pumped into the device when valve 14 is closed, a rise of pressure can be detected at measuring device 6. If the pressure at measuring device 6 has reached a defined value, valves 8 and 4 are closed and conveyors 9 and 3 are stopped.

Once the measured pressure value has stabilized and the system has settled, an evaluation period begins in which the stability of the built-up pressure is monitored. If the pressure falls below a defined value within this defined period (see dotted line in FIG. 5), the heat exchanger 13 is classified as defective (dashed line in FIG. 5). The maximum derivative of the measured pressure signal can serve as an indicator of the size of the leakage and can be related to the size of the leak.

This can be done using logic or a lookup table stored in the dialysis machine. This may also be based on a calculation as long as the pressure is measured continuously and the temperature is known.

While the above example is described in relation to the pressure value, the procedure can alternatively be carried out with the amount of the pressure value. For example, the pressure signal can correlate with the pressure value or the pressure signal can correlate with the amount of the pressure value. For example, similar signal curves could then be observed during pressure build-up and negative pressure build-up.

In the example shown in FIG. 2, the pressure sensor 6 is located between the rinsing bridge 5 and the dialyzer outlet valve 7. However, the pressure sensor can also be located at other points, for example downstream of the balancing device 10 and the conveying device 11 in line 12 closer to the heat exchanger 13.

In another example, pressure can be built up with one of the two concentrate pumps 3a, 3b when valves 2, 7 and 8 are closed. If no pressure can be built up in a predefined time or if it drops too quickly after the pump stops, this also indicates a leak in the heat exchanger 13.

In another example, pressure can be built up with one of the two concentrate pumps 3a, 3b when valves 2, 14, 4 and 8 are closed. If a rise of pressure is registered at pressure sensor 6 when valve 7 is open, this also indicates a leak in heat exchanger 13.

FIG. 3 shows a system according to the present disclosure. The illustrated arrangement avoids internal installation of the valve 14 within the dialysis machine 101, which may be desirable for product cost reasons. The basic method corresponds to FIG. 2, however, with the difference that valve 14 is designed to be portable and can, for example, be hosed in by a service technician at a STK or is designed as a pinch valve so that it is only used temporarily. For this purpose, valve 14 is equipped with a power supply device 17 and a synchronization device 16 so that the correct time point for closing can be communicated to valve 14 by the dialysis machine during a test. This can be done by wireless or wired communication 18 if the dialysis machine 101 is equipped with a corresponding communication device (not shown).

FIG. 4 shows an arrangement that avoids internal installation of the valve 14 within the dialysis machine 101, which may be desirable for product cost reasons. However, the valve is not controlled directly by the dialysis machine 101, but the outlet in the dialysis machine and the correct switching times for valve 14 are synchronized with the help of a control device 19. The control device can, for example, be the PC of a service technician who controls both the dialysis machine and valve 14 etc. by using maintenance software. Alternatively, the control device 19 can, for example, be part of a production infrastructure just like valve 14.

Although the present disclosure is illustrated and described in detail in the drawings and the foregoing description, these illustrations and descriptions are to be considered exemplary and not limiting. The present disclosure is not limited to the disclosed embodiments. In view of the foregoing description and drawings, it will be apparent to those skilled in the art that various modifications can be made within the scope of the present disclosure.

### Reference signs

1 pipe inlet side

2 Valve water inlet

3 Flow pump input

3a, 3b Concentrate pumps

4 Valve dialyzer inlet

5 Rinsing bridge

6 Pressure sensor

7 Dialyzer outlet valve

8 Bypass valve

9 Flow pump outlet

10 Balancing device

11 Conveyor

12 Water outlet pipe

13 Heat exchanger

14 Outlet valve

15 Drain pipe (outside)

16 Synchronization device

17 Energy supply device

18 Communication

19 Control device

20 Fluid circuit

20a first section

20b second section

21 Computing system

100 Blood treatment device

101 Dialysis machine

200 System

1. A method for leak testing of a heat exchanger of a blood treatment device, the method comprising the steps of:
monitoring a time curve of a pressure recorded by a pressure sensor in a first section of a fluid circuit of the blood treatment device in an evaluation period;
checking, by a computing system, whether the time curve of the pressure in the evaluation period fulfills one or more predetermined criteria; and
determining a leak of the heat exchanger when the time curve fulfills the one or more predetermined criteria,
the heat exchanger having two flow paths comprising a first flow path fluidically connected to the first section of the fluid circuit and a second flow path fluidically connected to a second section of the fluid circuit, and the fluid circuit connected to an inlet and an outlet via the flow paths,
the first section of the fluid circuit fluidically isolated from the second section of the fluid circuit and/or the inlet and/or the outlet during the evaluation period using one or more switching elements, and
the monitoring of the time curve in the evaluation period comprising monitoring the time curve in a first evaluation period during a conveyor operation for pressure build-up/negative pressure build-up in the first section and/or the second section of the fluid circuit and/or wherein the monitoring of the time curve in a second evaluation period comprises monitoring the time curve after a pressure build-up/negative pressure build-up in the first section of the fluid circuit.

2. The method according to claim 1, wherein at least one of:
the first section of the fluid circuit is fluidically separated from the second section of the fluid circuit and the outlet in the evaluation period using one or more switching elements,
the monitoring is carried out in the second evaluation period and the one or more predetermined criteria comprises that a rise of pressure/loss of pressure of the recorded pressure in the second evaluation period deviates from a reference rise of pressure/reference loss of pressure, or
the monitoring in the first evaluation period during a conveyor operation of a first conveyor arranged in the first section and/or a second conveyor for pressure build-up/negative pressure build-up in the first section, and the one or more predetermined criteria comprises that a rise of pressure/loss of pressure of the recorded pressure in the first evaluation period deviates from a reference rise of pressure/reference loss of pressure.

3. The method according to claim 2, wherein the first section is arranged downstream with respect to the second section.

4. The method according to claim 2, wherein the conveyor is arranged upstream or downstream with respect to the pressure sensor and/or wherein the first conveyor is arranged downstream with respect to the pressure sensor.

5. The method according to claim 1, wherein:
the first section of the fluid circuit is fluidically separated from the second section of the fluid circuit and the outlet in the evaluation period using one or more switching elements,
the monitoring is performed in the first evaluation period during a conveyor operation of a second conveyor arranged in the second section for pressure build-up/negative pressure build-up in the second section, and
the one or more predetermined criteria comprises that a rise of pressure/loss of pressure of the recorded pressure in the first evaluation period deviates from a reference rise of pressure/reference loss of pressure.

6. The method according to claim 5, wherein the first section is arranged downstream with respect to the second section.

7. The method according to claim 5, wherein the conveyor is arranged upstream or downstream with respect to the pressure sensor and/or wherein the first conveyor is arranged downstream with respect to the pressure sensor.

8. The method according to claim 5, wherein the one or more predetermined criteria comprises that the rise of pressure/loss of pressure is greater than the reference rise of pressure/reference loss of pressure.

9. The method according to claim 1, wherein at least one of:
the first section of the fluid circuit is fluidically separated from the second section of the fluid circuit and the inlet in the evaluation period using one or more switching elements, and in particular the second section is fluidically connected to the outlet,
the monitoring is carried out in the second evaluation period and the one or more predetermined criteria comprises that a rise of pressure/loss of pressure of the recorded pressure in the second evaluation period deviates from a reference rise of pressure/reference loss of pressure, or
the monitoring is performed in the first evaluation period during a conveyor operation of a second conveyor arranged in the first section for pressure build-up/negative pressure build-up in the first section, and the one or more predetermined criteria comprises that a rise of pressure/loss of pressure of the recorded pressure in the first evaluation period deviates from a reference rise of pressure/reference loss of pressure.

10. The method according to claim 9, wherein the first section is arranged upstream with respect to the second section and/or wherein the second conveyor is arranged upstream with respect to the pressure sensor.

11. The method according to claim 1, wherein the conveyor operation is set in the second evaluation period.

12. The method according to claim 1, wherein the second conveyor comprises one or more concentrate pumps configured to feed concentrate into a fluid in the fluid circuit.

13. The method according to claim 1, wherein the first section or the second section are fluidically separatable from the outlet by an external barrier element.

14. The method according to claim 13, wherein a time point for opening and/or closing the external barrier element is communicated from the blood treatment device to the external barrier element by means of a synchronization device.

15. A blood treatment device comprising:
a fluid circuit and a heat exchanger, wherein the heat exchanger has two flow paths comprising a first flow path fluidically connected to a first section of the fluid circuit and a second flow path fluidically connected to a second section of the fluid circuit, and the fluid circuit is connected to an inlet and an outlet via the flow paths;
a pressure sensor configured to detect a pressure in the first section of the fluid circuit of the blood treatment device; and
a computing system adapted for monitoring a time curve of the pressure recorded by the pressure sensor in an evaluation period in which the blood treatment device has a test configuration, for checking whether the time curve of the pressure in the evaluation period satisfies one or more predetermined criteria, and for determining a leak of the heat exchanger when the time curve satisfies the one or more predetermined criteria,
the first section of the fluid circuit in the test configuration being fluidically separated from the second section of the fluid circuit and/or the inlet and/or the outlet using one or more switching elements, and
monitoring the time curve in the evaluation period comprises monitoring the time curve in a first evaluation period during a conveyor operation for pressure build-up/negative pressure build-up in the first section and/or the second section of the fluid circuit and/or monitoring the time curve in a second evaluation period after a pressure build-up/negative pressure build-up in the first section of the fluid circuit.

16. The blood treatment device according to claim 15,
wherein at least one of:
in a first test configuration of the blood treatment device, the first section of the fluid circuit is fluidically separated from the second section of the fluid circuit and the outlet using one or more switching elements,
the blood treatment device is designed to perform the monitoring in the second evaluation period, and the one or more predetermined criteria comprises that a rise of pressure/loss of pressure of the recorded pressure in the second evaluation period deviates from a reference rise of pressure/reference loss of pressure, or
the blood treatment device is adapted to perform the monitoring in the first evaluation period during a conveyor operation of a first conveyor device arranged in the first section and/or a second conveyor device for pressure build-up/negative pressure build-up in the first section, and the one or more predetermined criteria comprises that a rise of pressure/loss of pressure of the recorded pressure in the first evaluation period deviates from a reference rise of pressure/reference loss of pressure.

17. The blood treatment device according to claim 15, wherein:
in a second test configuration of the blood treatment device, the first section of the fluid circuit is fluidically separated from the second section of the fluid circuit and the outlet using one or more switching elements, and
the blood treatment device is adapted to perform the monitoring in the first evaluation period during a conveyor operation of a second conveyor arranged in the second section for pressure build-up/negative pressure build-up in the second section and the one or more predetermined criteria comprises that a rise of pressure/loss of pressure of the recorded pressure in the first evaluation period deviates from a reference rise of pressure/reference loss of pressure.

18. The blood treatment device according to claim 15, wherein at least one of:
in a third test configuration of the blood treatment device, the first section of the fluid circuit is fluidically separated from the second section of the fluid circuit and the inlet using one or more switching elements and, in particular, the second section is fluidically connected to the outlet,
the blood treatment device is designed to perform the monitoring in the second evaluation period, and the one or more predetermined criteria comprises that a rise of pressure/loss of pressure of the recorded pressure in the second evaluation period deviates from a reference rise of pressure/reference loss of pressure, or
the blood treatment device is adapted to perform the monitoring in the first evaluation period during a conveyor operation of a second conveyor arranged in the first section for pressure build-up/negative pressure build-up in the first section and the one or more predetermined criteria comprises that a rise of pressure/loss of pressure of the recorded pressure in the first evaluation period deviates from a reference rise of pressure/reference loss of pressure.

19. A system comprising:
the blood treatment device according to claim 15;
an external barrier element;
a synchronization device; and
at least one of:
a control device of the blood treatment device, or
an external control device,
the external barrier element configured to fluidically separate the first section or the second section of the blood treatment device from the outlet, and
the blood treatment device configured to communicate a time point for opening and/or closing the external barrier element to the external barrier element via the synchronization device.
